(19) United States
(12) Patent Application Publication
(10) Pub. No.: US 2006/0105356 A1
(43) Pub. Date: May 18, 2006

(54) Methods for reducing evaporation in wet processing steps

(76) Inventors: Duane M. Dutton, San Jose, CA (US); Brent T. Tolosko, Santa Clara, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/992,889

(22) Filed: Nov. 18, 2004

(51) Int. Cl. C12Q 1/68; C12M 1/34; B05D 3/04; B05D 3/00

(52) U.S. Cl. 435/6; 427/377; 435/287.2; 427/2.11

(57) ABSTRACT

Methods of contacting a surface of an object with a liquid are provided. Aspects of the invention include positioning an object in a reaction chamber and flowing a liquid across a surface of the object, where at least a portion of the flowing step occurs in the presence of a gas that limits evaporation of a solvent component of the liquid. Also provided are devices for practicing the subject methods.

### BACKGROUND OF THE INVENTION

Contacting a liquid with a surface of a substrate, e.g., as is done in a wet processing protocol, is used in a variety of different applications. In many of these wet processing protocols, the liquid contacted with the substrate surface includes an agent in a solvent. Wet processing may be used in substrate coating processes in which a liquid such as, e.g., paint, ink, resist, etc., is contacted with a substrate surface to provide a coating on the surface. Wet processing may also be employed in chemical processing protocols in which a chemical process is performed on a substrate surface, for example in the fabrication of chemical arrays, e.g., nucleic acid chemical arrays.

Chemical arrays of nucleic acids have become an increasingly important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like.

A feature of many chemical arrays that have been developed is that each of the distinct nucleic acids of the array is stably attached to a discrete location on the array surface, such that its position remains constant and known throughout the use of the array. Stable attachment is achieved in a number of different ways, including covalent bonding of the polymer to the support surface and non-covalent interaction of the polymer with the surface.

There are two main ways of producing nucleic acid arrays in which the immobilized nucleic acids are covalently attached to the substrate surface, i.e., via in situ synthesis in which the nucleic acid ligand is grown on the surface of the substrate in a step-wise fashion and via deposition of the full ligand, e.g., a presynthesized nucleic acid/polypeptide, cDNA fragment, etc., onto the surface of the array.

Where the in situ synthesis approach is employed, conventional phosphoramidite synthesis protocols may be used. In phosphoramidite synthesis protocols, the 3′-hydroxyl group of an initial 5′-protected nucleoside is first covalently attached to the polymer support, e.g., a planar substrate surface. Synthesis of the nucleic acid then proceeds by deprotection of the 5′-hydroxyl group of the attached nucleoside, followed by coupling of an incoming nucleoside-3′-phosphoramidite to the deprotected 5′ hydroxyl group (5′-OH). The resulting phosphite triester is finally oxidized to a phosphotriester to complete the internucleotide bond. The steps of deprotection, coupling and oxidation are repeated until a nucleic acid of the desired length and sequence is obtained. Optionally, a capping reaction may be used after the coupling and/or after the oxidation to inactivate the growing DNA chains that failed in the previous coupling step, thereby avoiding the synthesis of inaccurate sequences.

In the synthesis of nucleic acids on the surface of a substrate, reactive deoxynucleoside phosphoramidites are successively applied, in molecular amounts exceeding the molecular amounts of target hydroxyl groups of the substrate or growing oligonucleotide polymers, to specific regions or cells of the high-density array, where they chemically bond to the target hydroxyl groups. Then, unreacted deoxynucleoside phosphoramidites from multiple cells of the high-density array are washed away, oxidation of the phosphite bonds joining the newly added deoxynucleosides to the growing oligonucleotide polymers to form phosphate bonds is carried out, and unreacted hydroxyl groups of the substrate or growing oligonucleotide polymers are chemically capped to prevent them from reacting with subsequently applied deoxynucleoside phosphoramidites. Optionally, the capping reaction may be done prior to oxidation.

Regardless of the particular wet processing protocol, a goal of a wet processing protocol is to provide a coating or chemical process that is uniform and defect-free.

As wet processing continues to be used in a variety of applications, there continues to be an interest in the development of improved wet processing protocols.

### SUMMARY OF THE INVENTION

Methods of contacting a surface of an object with a liquid are provided. Aspects of the invention include positioning an object in a reaction chamber and flowing a liquid across a surface of the object, where at least a portion of the flowing step occurs in the presence of a gas that limits evaporation of a solvent component of the liquid.

Also provided are methods of producing a nucleic acid array where embodiments include positioning a substrate, that includes a surface displaying a covalently bound blocked nucleotide, in a flow cell; flowing a liquid that includes a deblocking reagent in a solvent across the surface in a flow cell to deblock the bound nucleotide, wherein the liquid is flowed across the surface by introducing a volume of the liquid into the flow cell and then removing the volume of liquid from the flow cell, wherein at least a portion of the flowing occurs in the presence of a gas that limits evaporation of the solvent; contacting the bound deblocked nucleotide with a blocked nucleoside to covalently bond the second blocked nucleoside to the bound deblocked nucleotide; and reiterating the positioning and flowing steps at least once to produce a nucleic acid.

Also provided are apparatuses for practicing the subject methods. Embodiments include an apparatus for producing a chemical array and include a chemical moiety precursor deposition element for depositing a chemical moiety precursor into a surface of a substrate; a reaction chamber for flowing a liquid across a surface if a substrate in communication with a reservoir the liquid, wherein the liquid includes a functional group generation agent in a solvent; and a reservoir of a gas in fluid communication with the reaction chamber, wherein the reservoir includes a gas that limits evaporation of the solvent.

### BRIEF DESCRIPTIONS OF THE DRAWINGS

FIG. 1 shows an exemplary substrate carrying an array, such as may be fabricated in accordance with the subject methods.

FIG. 2 shows an enlarged view of a portion of FIG. 1 showing spots or features.

FIG. 3 is an enlarged view of a portion of the substrate of FIG. 1.

FIG. 4 shows an exemplary substrate, such as may be used in the methods of the subject invention.

FIG. 5 is a schematic diagram depicting an embodiment of an apparatus for conducting synthesis of arrays according to an embodiment of the subject invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than about 10 to about 20 residues. The terms “polypeptide” and “protein” may be used interchangeably.

The term “protein” as used herein refers to polypeptides of specific sequence of more than about 50 residue and includes D and L forms, modified forms, etc.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source. A “biomonomer” references a single unit, which can be linked with the same or other biomonomers to form a biopolymer (e.g., a single amino acid or nucleotide with two linking groups, one or both of which may have removable protecting groups).

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

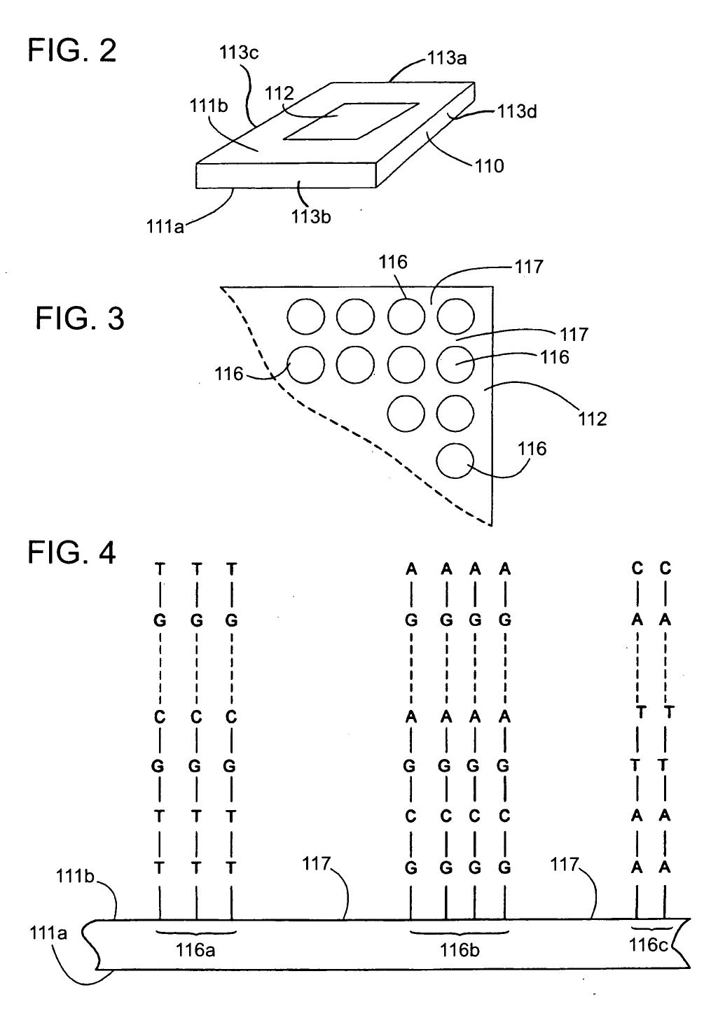

An exemplary chemical array is shown in FIGS. 2-4, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), and other polynucleotides which are C-glycosides of a purine or pyrimidine base. In the practice of the instant invention, oligomers will generally comprise about 2-60 monomers, preferably about 10-60, more preferably about 50-60 monomers.

“Activator” refers to any suitable chemical and/or physical entity that is employed to make-possible, assist, enhance or increase in the joining or linking of a monomer to another chemical entity such as one or more other monomers or a reactive functional group such as a free hydroxy functional group present on a substrate surface, etc. For example, an activator may protonate a monomer so that it may be joined to another monomer or to a free functional group. For example, activators may be employed in phosphoramidite chemistry where they used in the joining of a deoxynucleoside phosphoramidite to a functional group present on a substrate surface or to another deoxynucleoside phosphoramidite. In producing nucleic acids on a substrate surface using phosphoramidite chemistry, one of the first steps in such a protocol involves attaching a first monomer to the substrate surface. Accordingly, a solution containing a protected deoxynucleoside phosphoramidite and an activator, such as tetrazole, benzoimidazolium triflate (“BZT”), S-ethyl tetrazole, and dicyanoimidazole, is applied to the surface of a substrate that has been chemically prepared to present reactive functional groups such as, for example, free hydroxyl groups. The activators tetrazole, BZT, S-ethyl tetrazole, and dicyanoimidazole are acids that protonate the amine nitrogen of the phosphoramidite group of the deoxynucleoside phosphoramidite. A free hydroxyl group on the surface of the substrate displaces the protonated secondary amine group of the phosphoramidite group by nucleophilic substitution and results in the protected deoxynucleoside covalently bound to the substrate via a phosphite triester group. An analogous methodology using an activator may be employed to link two deoxynucleoside phosphoramidites together such as a deoxynucleoside phosphoramidite to a substrate bound nucleotide. For example, a protected deoxynucleoside phosphoramidite in solution with an activator is applied to the substrate-bound nucleotide and reacts with the 5′ hydroxyl of the nucleotide to covalently link the protected deoxynucleoside to the 5′ end of the nucleotide via a phosphite triester group. In accordance with the subject invention, suitable “activators” include, but are not limited to, tetrazole and tetrazole derivatives such as S-ethyl tetrazole, dicyanoimidazole (“DCI”), benzimidazolium triflate (“BZT”), and the like. Activators are usually, though not always, present in a liquid, typically in solution, where such may be referred to as a “fluid activator”. In describing the subject invention, an activator includes an activator alone or with a suitable medium such as a fluid medium or the like. As such, an activator and a fluid activator may be used interchangeably herein.

The term “sample” as used herein relates to a material or mixture of materials, typically, although not necessarily, in fluid form, containing one or more components of interest.

The terms “protection” and “deprotection” as used herein relate, respectively, to the addition and removal of chemical protecting groups using conventional materials and techniques within the skill of the art and/or described in the pertinent literature; for example, reference may be had to Greene et al., Protective Groups in Organic Synthesis, 2nd Ed., New York: John Wiley & Sons, 1991. Protecting groups prevent the site to which they are attached from participating in the chemical reaction to be carried out.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier may be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position, place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information references transmitting the data representing that information as signals (e.g., electrical, optical, radio signals, etc.) over a suitable communication channel (e.g., a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that many computer-based systems are available which are suitable for use in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

Items of data are “linked” to one another in a memory when the same data input (for example, filename or directory name or search term) retrieves the linked items (in a same file or not) or an input of one or more of the linked items retrieves one or more of the others.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

### DETAILED DESCRIPTION OF THE INVENTION

Methods of contacting a surface of an object with a liquid are provided. Aspects of the invention include positioning an object in a reaction chamber and flowing a liquid across a surface of the object, where at least a portion of the flowing step occurs in the presence of a gas that limits evaporation of a solvent component of the liquid. Also provided are devices for practicing the subject methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The figures shown herein are not necessarily drawn to scale, with some components and features being exaggerated for clarity.

As summarized above, the subject invention includes contacting an agent with a surface of an object and particularly contacting a liquid with an object where the liquid includes the agent in a solvent. The liquid that includes the agent in a solvent is contacted with the surface of the object in an environment that limits the evaporation of the solvent from the liquid. Embodiments include environments such as reaction chambers, e.g., flow cells, which include a gas that limits the evaporation of the solvent from the liquid. As such, aspects of the subject methods include contacting an object with a liquid, where in the liquid includes an agent in a solvent, in a reaction chamber that includes a gas that limits the evaporation of the solvent from the liquid. At least some of the time that the liquid is in the reaction chamber, the reaction chamber also includes the evaporation-limiting gas. For example, the gas may be present in the reaction chamber at least during the removal of the liquid from the reaction chamber, and in certain embodiments at all times that the liquid is present in the reaction chamber.

The subject invention may be used in a variety of applications in which an agent is contacted with an object in a reaction chamber, where applications include, but are not limited to, coating applications and chemical processing applications and may be used, e.g., in semiconductor fabrication (e.g., contacting an etchant or developer to an object), chemical array fabrication, and the like. It is to be understood that the description of the subject invention with respect to producing a chemical array is exemplary only and in no way intended to limit the scope of the invention. For example, the subject invention is described below primarily with respect to limiting evaporation of a solvent used in a functional group generation protocol in a flow cell, where such is exemplary only and in no way intended to limit the scope of the invention. It will be apparent that the subject invention may be used in a variety of protocols including protocols other than chemical array fabrication protocols.

Embodiments of the subject methods include positioning an object in a reaction chamber. A reaction chamber may generally be characterized as a housing having a cavity or housing chamber therein configured to receive an object. The reaction chamber may have a variety of different dimensions and configurations, where the particular configuration chosen will depend, at least in part, on the manner in which fluid is contacted with the object in the reaction chamber. For example, the reaction chamber may be configured to contact fluid with an object therein in a number of different manners, including, but not limited to: flowing liquid across the surface of an object, submerging an object in a liquid, spraying a liquid onto a surface, and the like. Reaction chambers therefore include, but are not limited to, flow cells as described herein. The reaction chamber may have one or more ports for introducing and/or removing one or more fluids into and/or from the reaction chamber, i.e., a fluid input port and a fluid output port, where the same port may be used for both input and output in certain embodiments. For example, a reaction chamber may include one or more ports for introducing a liquid that includes an agent in a solvent to the chamber to contact the liquid with a surface of an object positioned within the reaction chamber and for removing the liquid from the reaction chamber. One or more ports may also be included for introducing an evaporation-limiting gas to the reaction chamber so that the gas is present in at least a portion of the chamber when the liquid is flowed across the surface of the object, and for removing the gas from the chamber.

As described above, in certain embodiments a reaction chamber is a flow cell. A flow cell may be described broadly as having a housing that forms a chamber where an object such as an array substrate may be positioned. As summarized above, the flow cell allows fluids to be passed through the flow cell chamber where the object such as the array substrate is disposed. The array substrate may be mounted in the chamber in or on a holder. The flow cell housing usually further includes at least one fluid inlet and at least one fluid outlet for flowing fluids into and through the chamber in which the support is mounted. In one approach, the fluid outlet may be used to vent the interior of the reaction chamber for introduction and removal of fluid by means of the inlet. On the other hand, fluids may be introduced into the reaction chamber by means of the inlet with the outlet serving as a vent and fluids may be removed from the reaction chamber by means of the outlet with the inlet serving as a vent.

The dimensions of the housing chamber of the employed flow cell may vary and are dependent on the dimensions of the support that is to be placed therein. In certain embodiments, the array substrate may be one on which a single array of chemical compounds is synthesized. In this regard the substrate may range from about 1.5 to about 5 inches in length and about 0.5 to about 3 inches in width. The substrate may range from about 0.1 to about 5 mm, e.g., about 0.5 to about 2 mm, in thickness. A standard size microscope slide is usually about 3 inches in length and 1 inch in width and may be used. Alternatively, multiple arrays of chemical compounds may be synthesized on a given substrate or wafer, which may be used as is or which may then be diced, i.e., cut, into single array substrates in which each dices section may include one or more chemical arrays. In this alternative approach the substrate may range from about 5 to about 8 inches in length and about 5 to about 8 inches in width so that the substrate may be diced into multiple single array substrates having the aforementioned dimensions. The thickness of the substrate may be the same as that described above. In a specific embodiment by way of illustration and not limitation, a substrate that is about 65/8 inches by about 6 inches may be employed and diced into about 1 inch by about 3 inch substrates.

Representative flow cells that may be employed in certain embodiments may be about 6.5 inches wide by about 6 inches tall in the plane of the flow cell. More generally these dimensions may range from the size of an array about 1 cm square to about 1 meter square. The gap width in representative embodiments of flow cells that may be employed in the invention may range from about 1 μm to about 500 μm, and in certain embodiments may range from about 1-10 μm to about 10 mm.

Flow cell devices employed in array fabrication which may be adapted for use with the subject invention are further described in, for example, U.S. Published Patent Application Nos. 20040180450; 20030003222; 20030003504; 20030112022; 200030228422; 200030232123; and 20030232140; and U.S. Pat. No. 6,713,023.

The housing of the flow cell is generally constructed to permit access into the chamber therein. The flow cell may have an opening that is sealable to fluid transfer after the array substrate is placed therein. Such seals may include a flexible material that is sufficiently flexible or compressible to form a fluid tight seal that may be maintained under increased pressures encountered in the use of the device. The flexible member may be, for example, rubber, flexible plastic, flexible resins, and the like and combinations thereof. In any event the flexible material should be substantially inert with respect to the fluids introduced into the device and must not interfere with the reactions that occur within the device. The flexible member may be a gasket and may be in any shape such as, for example, circular, oval, rectangular, and the like, e.g., the flexible member may be in the form of an O-ring in certain embodiments.

Alternatively, the housing of the flow cell may be conveniently constructed in two parts, which may be referred to generally as top and bottom elements. These two elements are sealably engaged during synthetic steps and are separable at other times to permit the support to be placed into and removed from the chamber of the flow cell. Generally, the top element is adapted to be moved with respect to the bottom element although other situations are contemplated herein. Movement of the top element with respect to the bottom element may be achieved by means of, for example, pistons, and so forth. The movement may be controlled electronically by means that are conventional in the art. In another approach a reagent chamber may be formed in situ from an array substrate and a sealing member. The inlet of the flow cell is usually in fluid communication with an element that controls the flow of fluid into the flow cell such as, for example, a manifold, a valve, and the like or combinations thereof. This element, in turn, is in fluid communication with one or more fluid reagent dispensing stations. In this way different fluid reagents for one step in the synthesis of the chemical compound may be introduced sequentially into the flow cell.

In one embodiment, the fluid dispensing stations may be affixed to a base plate or main platform to which the flow cells are mounted. Any fluid dispensing station may be employed that dispenses fluids such as water, aqueous media, organic solvents, ionic liquids, gases, and the like. The fluid dispensing station may include a pump for moving fluid and may also comprise a valve assembly and a manifold as well as a means for delivering predetermined quantities of fluid to the flow cell. The fluids may be dispensed by pumping from the dispensing station. In this regard, any standard pumping technique for pumping fluids may be employed in the present apparatus. For example, pumping may be by means of a peristaltic pump, a pressurized fluid bed, a positive displacement pump, e.g., a syringe pump, and the like.

The object may be any suitable object that has a surface that requires contact with an agent. In certain embodiments, the object is a substrate having a planar surface. In certain embodiments, the substrate is an array substrate, i.e., a substrate upon which one or more chemical arrays may be carried. The substrate may be glass such as flat glass whose surface has been chemically activated to support binding or synthesis thereon, glass available as Bioglass and the like. However, the substrate may be made from materials such as inorganic powders, e.g., silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; ceramics, metals, and the like.

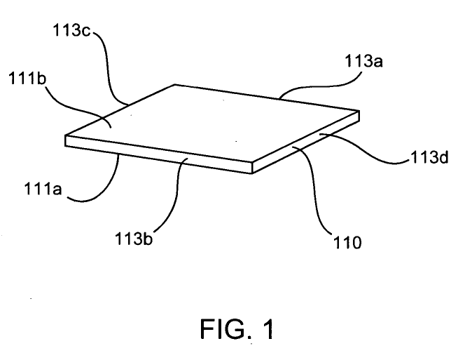

An exemplary object is shown in FIG. 1, where the object shown in this embodiment includes a contiguous planar substrate 110 having a front surface and a rear surface 111b. Such a substrate may be used to carry one or more arrays, e.g., on rear surface 111b (see for example FIGS. 2-4). Substrate 110 includes opposed sides 113c, 113d and leading end 113a and trailing end 113b. Although substrate 110 is shown as having rectangular shape, it is to be understood that substrate 110 may be of any shape.

A variety of agents may be contacted to a surface of the object, where the particular agent will depend on the particular application. In certain embodiments as described below, the agent is an agent used in an array synthesis protocol such as a functional group generations agent such as a capping agent, deblocking agent (e.g., a species of chloroacetic acid such as dichloroacetic acid), oxidizing agent (e.g., iodine (I₂)), and the like, as described in greater detail below.

Regardless of the agent, in contacting the agent with an object, the agent is in a solvent such that a liquid that includes the agent in a solvent is contacted with an object. That is, any reagent that is normally a solid reagent may be converted to a liquid reagent by dissolution in a suitable solvent, which may be a protic solvent or an aprotic solvent. The solvent may be an aqueous medium that is solely water or may contain a buffer, or may contain from about 0.01 to about 80 or more volume percent of a cosolvent such as an organic solvent as mentioned above. The solvent may, in certain embodiments, be an ionic liquid. The particular solvent will depend of course on the agent used, where in certain embodiments the solvent is a volatile solvent. Aspects include, but are not limited to, solvents that have a vapor pressure ranging from about 0.3 mm to about 400 mm at 20° C. and 760 mm pressure. as determined at standard temperature and pressure conditions i.e., STP conditions (0° C.; 1 ATM). The solvent may be an organic solvent or an inorganic solvent, where exemplary solvents include, but are not limited to, acetonitrile, tetrahydrofuran (THF), pyridine, toluene, xylene (o, m, p), ethylbenzene, perfluoro-n-heptane, perfluoro decalin, chlorobenzene, 1,2 dichloroethane, 1,1,2 trichloroethane, 1,1,2,2 tetrachloroethane, pentachloroethane, ethyl ether, propyl ether, dimethylformamide, dimethylsulfoxide, acetone, methanol, ethanol, trimethylbenzene, etc. Accordingly, the subject invention provides methods for limiting evaporation of the solvent from the liquid, e.g., by employing an evaporating-limiting gas at least during part of the time the liquid is contacted with the substrate, e.g., at when the liquid is removed from the reaction chamber in which the substrate is positioned.

Once an object is suitably positioned in a reaction chamber, the liquid that includes the agent in the solvent is contacted with a surface of the object in a manner that limits the evaporation of the solvent from the liquid. The liquid may be contacted with the object in any suitable manner. For example, the liquid may be flowed across the surface of the substrate, e.g., introduced into the reaction chamber via a liquid introduction port in a manner so that the fluid is flowed across at least a portion of a surface of the object and removed (e.g., drained) from the reaction chamber after so-flowed via the same or different port, e.g., removed through a dedicated liquid removal port.

The evaporation of the solvent of the liquid is limited by providing a reaction chamber environment that limits the evaporation of the solvent, where in certain embodiments the environment is a gaseous environment. As such, embodiments include introducing a gas to the reaction chamber during at least a portion of the time the liquid is present in the reaction chamber, e.g., during at least the time the liquid is being flowed across the surface of the object. Accordingly, embodiments include flowing a liquid across a surface of an object, in a reaction chamber, at least partially in the presence of a gas that limits the evaporation of the solvent from the liquid.

The gas of an evaporation-limiting environment of the reaction chamber may be any suitable gas, where in certain embodiments the gas is one that is inert with respect to at least the agent of the liquid. In other words, for the period of time the liquid is in contact with the gas in the reaction chamber, the gas is unreactive with respect to the agent of the liquid. In certain embodiments, the gas includes at least two components, where the first component is a gas, e.g., that is inert to the agent, and the second component is one that serves to maintain the concentration of the agent in the liquid contacting the surface of the object in a desired range. In certain embodiments, the second component may be the agent itself, e.g., where the active agent itself is volatile. In yet other embodiments, the second component is a solvent, where the solvent component is the same solvent that is also present in the liquid such that the liquid includes a solvent and the gas includes the same solvent. In yet other embodments, the gas may include both agent and solvent. The gas may be any suitable gas, where in certain embodiments the gas may be N₂, Ar, and the like.

The amount of solvent in the gas is sufficient to limit the evaporation of the solvent of the liquid. In certain embodiments, the gas present in the reaction chamber includes a sufficient concentration of solvent such that the solvent is in equilibrium between the gas and any liquid present on the surface of the object. For example, the gas may include a sufficient amount of solvent so that the liquid does not lose vapor molecules to the gas by vaporization. Aspects of the subject invention include a gas that is saturated with solvent.

The solvent may be added to the gas in any suitable manner, e.g., a gas may be saturated with a solvent by bubbling, misting, spraying, and diffusion through a semi-permeable membrane. For example, certain embodiments include bubbling a gas (e.g., nitrogen) through a finely fritted or porous structure into the solvent in a manner so that a gas mixture (gas and solvent mixture) has the same proportion of solvent as the liquid that includes the agent in the solvent. In certain embodiments, the bubbles of the gas are small bubbles, e.g., having a mean diameter less than about 100 μm, e.g., less than about 80 μm, e.g., less than about 70 μm. In certain embodiments the gas bubbles may travel through a tortuous path, e.g., spiral path or the like, so that the bubbles may have sufficient contact with the solvent to transfer a saturated vapor into the traveling gas bubbles. In alternative embodiments, a gas and the solvent to be added to the gas may be introduced into a vessel which may be the reaction chamber or other vessel in communication with the reaction chamber. In this manner, vapor saturation of the gas may be achieved by introducing the gas through a port of the vessel by aspirating, carburating, or atomized mist injecting the solvent into the vessel, e.g., through another port. Any excess solvent may be collected from the vessel and re-injected into the vessel.

For clarity and ease of description, the invention is now described further in terms of an exemplary nucleic acid array fabrication embodiment, where such description is not intended to limit the scope of the invention.

It is to be understood that the subject invention may be employed in the fabrication of different chemical arrays, e.g., peptide arrays, and the like. Accordingly, the present methods and apparatus may be used in the synthesis of nucleic acid, e.g., oligonucleotide, arrays. The synthesis of arrays of polynucleotides on the surface of a support in certain approaches, e.g., in situ fabrication protocols, involves attaching an initial nucleoside or nucleotide to a functionalized surface. In one approach the surface is reacted with nucleosides or nucleotides that are also functionalized for reaction with the groups on the surface of the support. Methods for introducing appropriate amine specific or alcohol specific reactive functional groups into a nucleoside or nucleotide include, by way of example, addition of a spacer amine containing phosphoramidites, addition on the base of alkynes or alkenes using palladium mediated coupling, addition of spacer amine containing activated carbonyl esters, addition of boron conjugates, formation of Schiff bases.

As noted above, embodiments of the subject invention include methods of producing nucleic acid arrays by in situ synthesis of two or more distinct nucleic acids on the surface of a solid support. The in situ synthesis protocol employed in the subject invention may be viewed as an iterative process that includes two or more cycles, where each cycle includes the following steps: a monomer attachment step in which a blocked nucleoside monomer is covalently bonded to two or more distinct locations, e.g., at least a first and second location, of a functional group, e.g., hydroxyl-, amino-, etc., displaying surface of a solid support; and an internucleotide linkage stabilization and 5′ functional group generation step in which the phosphite triester linkage is oxidized and functional groups are generated at the blocked ends of the resultant attached blocked nucleotides by removal of the blocking groups for addition of subsequent nucleoside monomers.

In certain embodiments, each cycle includes the following steps: a monomer attachment step in which a 5′OH blocked nucleoside monomer is covalently bonded to two or more distinct locations, e.g., at least a first and second location, of a hydroxyl functional group displaying surface of a solid support, e.g., a surface of a solid support displaying hydroxyl functional groups or a surface displaying intermediate nucleic acids having 5′OH groups; and an internucleotide linkage stabilization and 5′OH generation step in which the phosphite triester linkage is oxidized and hydroxyl groups are generated at the 5′ ends of the resultant attached blocked nucleotides by removal of the blocking groups for addition of subsequent nucleoside monomers, where this step includes oxidizing and deblocking substeps, as well as optionally a capping step. These repetitive steps that may include washing, oxidation, deprotecting, blocking and optionally capping may be performed in a reaction chamber such as a flow cell.

Accordingly, embodiments include array in situ fabrication methods in which multiple different reagent droplets are deposited, e.g., by a fluid deposition device such as a pulse jet or other means, at a given target location in order to form the final feature (hence a probe of the feature is synthesized on the array substrate). Aspects thus include, at each of the multiple different addresses at which features are to be formed, the same conventional iterative sequence used in forming polynucleotides from nucleoside reagents on a substrate by means of known chemistry. This iterative sequence may be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. Coupling may be performed by depositing drops of an activator and phosphoramidite at the specific desired feature locations for the array. Capping, oxidation and deprotection may be accomplished by treating the entire substrate with a layer of the appropriate reagent such as sequentially flowing the particular reagent(s) across the substrate surface, for example in a reaction chamber such as a flow cell system. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases may be accomplished using alkaline conditions such as ammonium hydroxide, in another reagent contacting step such as described above in a reaction chamber such as a flow cell system. A single pulse jet or other dispenser may be assigned to deposit a single monomeric unit.

In situ fabrication protocols are known and include those described in U.S. Pat. No. 5,449,754 for synthesizing peptide arrays, and in U.S. Pat. No. 6,180,351 and WO 98/41531 and the references cited therein for polynucleotides. The chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643 and European patent application, EP 0294196, and elsewhere. The phosphoramidite and phosphite triester approaches are most broadly used, but other approaches include the phosphodiester approach, the phosphotriester approach and the H-phosphonate approach. The substrates may be functionalized to bond to the first deposited monomer. Suitable techniques for functionalizing substrates with such linking moieties are described, for example, in Southern, E. M., Maskos, U. and Elder, J. K., Genomics, 13, 1007-1017, 1992.

An exemplary in situ synthesis protocol is now described and specific exemplary steps of a representative synthesis cycle (monomer attachment and generation of functionalities) are now described separately in greater detail in terms of these particular embodiments. However, the scope of the invention is not so limited, as the invention being described in terms of these particular exemplary embodiments for ease of description only. Various modifications may be made and still fall within the scope of the invention. For example, the “direction” of synthesis may be reversed, such that the synthesized nucleic acids are attached to the substrate at their 5′ ends and one generates 3′ functional groups in the deblocking/deprotecting step and certain fluids may be omitted and/or certain fluids may be added to the sequence. The use of 5′OH functional groups, acid labile blocking groups, such as DMT and the use of an acid deblocking agent, are merely exemplary. Other functional groups may be employed, e.g., amine functional groups. In yet other embodiments, base labile blocking groups may be employed, where such groups and the use thereof are described in U.S. Pat. No. 6,222,030. In these latter types of embodiments, the acid deblocking agent described above may be replaced with a base deblocking agent.

Monomer Attachment Step

In the monomer attachment step of each cycle, one or more different 5′OH blocked nucleoside monomers is contacted with one or more different locations of a substrate surface that displays hydroxyl functional groups, such that the nucleoside monomers become covalently bound to the surface, e.g., via a nucleophilic substitution reaction between the an activated (e.g., protonated) phosphoramidite moiety of the blocked nucleoside monomer and the surface displayed hydroxyl functionality. The surface-displayed hydroxyl functionality may be on the surface of a nascent substrate, i.e., a substrate surface that not yet include deposited monomers, or may be at the 5′ end of a growing nucleic acid, depending on the particular point in the synthesis protocol. For example, at the beginning of a particular synthesis protocol, the surface-displayed hydroxyl functional groups are immediately on the surface of a solid support or substrate. In contrast, following one or more cycles of a given synthesis protocol, the surface displayed functional groups are present at the 5′ ends of growing nucleic acids which, in turn, are covalently bonded to the surface of the substrate.

As such, at the beginning of any array synthesis protocol, the first step is to provide a substrate having a surface that displays hydroxyl functional groups, where the hydroxyl functional groups are employed in the covalent attachment of the growing nucleic acid polymers on the substrate surface during synthesis. The substrate may be any convenient substrate that finds use in biopolymeric arrays. In general, the substrate may be rigid or flexible. The substrate may be fabricated from a variety of materials. In certain embodiments, the materials from which the substrate may be fabricated may exhibit a low level of non-specific binding during hybridization events. In many situations, it is of interest to employ a material that is transparent to visible and/or UV light. Specific materials of interest include: silicon; glass; plastics, e.g., polytetrafluoroethylene, polypropylene, polystyrene, polycarbonate, and blends thereof, and the like; metals, e.g. gold, platinum, and the like; etc. The surface may include one or more different layers of compounds that serve to modify the properties of the surface in a desirable manner. Such modification layers, when present, may range in thickness from a monomolecular thickness to about 1 mm, e.g., from a monomolecular thickness to about 0.1 mm or from a monomolecular thickness to about 0.001 mm. Modification layers of interest include: inorganic and organic layers such as metals, metal oxides, conformal silica or glass coatings, polymers, small organic molecules and the like. Polymeric layers of interest include layers of: peptides, proteins, nucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated. The particular surface chemistry will be dictated by the specific process to be used in polymer synthesis, as described in greater detail infra. However, as mentioned above, in one aspect, the substrate that is initially employed has a surface that displays hydroxyl functional groups.

In one aspect, nucleic acid arrays are produced by synthesizing nucleic acid polymers using conventional phosphoramidite solid phase nucleic acid synthesis chemistry where the solid support is a substrate as described above. Phosphoramidite-based chemical synthesis of nucleic acids is well known to those of skill in the art, being reviewed above and in U.S. Pat. Nos. 5,436,327 and 4,415,732.

To produce nucleic acid arrays according to the subject methods, a substrate surface as described above having the appropriate surface groups, e.g., —OH groups, present on its surface, is obtained. In one aspect, the synthesis protocol is carried out under anhydrous conditions and reactions are carried out in a non-aqueous, typically organic solvent layer on the substrate surface.

First residues of each nucleic acid to be synthesized are covalently attached to the substrate surface via reaction with the surface bound —OH groups. Depending on whether the first nucleotide residue of each nucleic acid to be synthesized on the array is the same or different, different protocols for this step may be followed. Where each of the nucleic acids to be synthesized on the substrate surface have the same initial nucleotide at the 3′ end, the entire surface of the substrate is contacted with the blocked, activated nucleoside under conditions sufficient for coupling of the activated nucleoside to the reactive groups, e.g., —OH groups, present on the substrate surface to occur. The entire surface of the array may be contacted with the fluid composition containing the activated nucleoside using any convenient protocol, such as flooding the surface of the substrate with the activated nucleoside solution, immersing the substrate in the solution of activated nucleoside, etc. The fluid composition typically includes a fluid composition of the blocked nucleoside in an organic solvent, e.g., acetonitrile, where the fluid composition may include an activating agent, e.g., tetrazole, benzoimidazolium triflate (“BZT”), S-ethyl tetrazole, and dicyanoimidazole, etc.

Alternatively, in one aspect, where the initial residue of the various nucleic acids differs among the nucleic acids, one or more sites on the substrate surface are individually contacted with a fluid composition of the appropriate blocked, activated nucleoside. Of particular interest in many embodiments is the use of pulse-jet deposition protocols, such as those described in U.S. Pat. Nos. 6,171,797; 6,180,351; 6,232,072; 6,242,266; 5,449,754; 6, 300,137; and 6,323,043; as well as U.S. patent application Ser. No. 09/302,898 filed Apr. 30, 1999. In one aspect, two or more different fluid compositions of activated, blocked nucleosides, which fluid compositions differ from each other in terms of the activated nucleoside present therein, are each pulse-jetted onto one or more distinct locations of the surface, where the type of fluid composition pulse-jetted at the locations is dictated by the sequence of the desired nucleic acid at each location.

In another aspect, the activated nucleoside monomers employed in this attachment step of each cycle of the subject synthesis methods are blocked at their 5′-OH functionalities (ends) with an acid labile blocking group. By acid labile blocking group is meant that the group is cleaved in the presence of an acid to yield a 5′-OH functionality. The acid labile blocking group may be DMT in certain embodiments.

The above step of the subject protocols results in a “blocked monomer attached substrate” where the surface of the substrate includes blocked monomers, e.g., DMT-blocked nucleoside monomers, covalently attached to the surface, either directly, if the blocked monomers are the first residues to be synthesized surface-bound nucleic acids, or indirectly, i.e., where blocked monomers are at the end of growing nucleic acid chains, in which case it may interchangeably be referred to as a polymer-attached substrate, where blocked monomer attached substrate is used herein for convenience. This resultant “blocked monomer attached substrate” is then subjected to the next step of the subject synthesis cycle, i.e., the 5′OH generation step.

Generation of Functionalities

As summarized above, following covalent attachment of activated, blocked nucleoside monomers to one or more locations of the substrate surface, functional, e.g., 5′OH, moieties are then generated on the surface so that the synthesis cycle can be repeated with a new round of activated, blocked nucleoside monomers. This generation step includes (in certain embodiments) the following substeps: (a) oxidation; (b) optional capping; and (c) deblocking, where the generation of functionalities may be performed using a flow cell or other analogous structure, in certain embodiments. Accordingly, for example, after addition of a nucleoside monomer, such as depositing the reagent using a pulse-jet method, the substrate may be placed into a chamber of a flow cell which allows one or more functional group generation liquids to be passed through the chamber, where the substrate is disposed at least partially in the presence of a gas that limits evaporation of a solvent of the liquid during functional group generation. Any suitable protocol may be used to flow one or more fluid across a substrate surface to generate a functional group on a chemical moiety, where methods for flowing one or more liquids across a surface of a substrate, e.g., by purging one liquid with another, and which may be adapted for use in the subject invention are described, e.g., U.S. patent application Ser. Nos. 10/813,331; 10/813,337; and 10/813,467.

In one embodiment, each of these substeps is accomplished by contacting the entire surface of the substrate with an appropriate liquid, i.e., an oxidation liquid, a capping liquid, a deblocking liquid, a wash liquid, etc. Contact of the entire surface is achieved in the subject methods by flooding the surface with the appropriate fluid, where a flow cell approach is employed in exemplary embodiments, such that the entire substrate is contacted with a volume of the appropriate liquid, e.g., by flowing a volume of the appropriate liquid over the surface of the substrate in an appropriate container or chamber, e.g., such as a flow cell. In one embodiment, performance of each substep includes flowing an adequate volume of the appropriate liquid over the substrate surface so that the entire surface of the substrate is contacted with the liquid.

In some embodiments, wash reagent is first allowed to pass into and out of the flow cell. Oxidizing agent present in an oxidizing liquid that includes a solvent may then be introduced into the flow cell. Following an additional wash step, the substrate surface may then be subjected to a deblocking step. In this step, a deblocking agent for removing a protecting group, in a deblocking liquid that includes a solvent, is flooded over the substrate surface. In one aspect, a wash liquid may then be flooded over the substrate. Optionally, the substrate surface may be contacted with a capping agent in a capping liquid that includes a solvent, where the surface may be contacted with a capping liquid at one or more times, e.g., prior to oxidation, prior to deblocking, etc.

In any of these steps, the flow cell in which the substrate is positioned includes a gas that limits the evaporation of the solvent of the particular liquid present in the reaction chamber. Since the liquids may be sequentially flowed across a substrate in flow cell one after the other, the gas present in the flow cell may change depending on the particular solvent of the liquid present in the flow cell at a given time. For example, a first liquid having a first agent in a first solvent may be flowed across a substrate at least partially in the presence of a first gas that limits the evaporation of the first solvent, which first liquid may be followed by a second liquid having a second agent in a second solvent flowed across the substrate in the presence of a second gas that limits evaporation of the second solvent, etc. Accordingly, different liquids (that include different solvents) and gases (that include an evaporation-limiting concentration of the solvent of the liquid) may be continually introduced and removed from a flow cell, e.g., in a sequential manner, so that evaporation of a solvent of functional group generation reagent present in the flow cell is limited, where such may be controlled by a processor as described below.

In any event, embodiments include flowing a liquid that includes a functional group generation reagent in a solvent across the surface of a substrate that includes a first chemical moiety precursor, in a flow cell, to generate a functional group on a chemical moiety. The liquid is flowed at least partially in the presence of a gas that limits evaporation of the solvent.

Representative deblocking, oxidation, capping and wash fluids are now described. It should be noted that the following descriptions of deblocking, oxidizing, capping and wash fluids are merely representative, and that other types of fluids may be employed in a given protocol, e.g., a combined oxidizing/deblocking fluid, such as that described in Published United States Application No. 20020058802.

Oxidation

Oxidation results in the conversion of phosphite triesters present on the substrate surface following coupling to phosphotriesters. Oxidation is accomplished by contacting the surface with an oxidizing solution, as described above, which solution includes a suitable oxidating agent. Various oxidizing agents may be employed, where representative oxidizing agents include, but are not limited to: organic peroxides, oxaziridines, iodine, sulfur etc. The oxidizing agent is typically present in a fluid solvent, where the fluid solvent may include one or more cosolvents, where the solvent components may be organic solvents, aqueous solvents, ionic liquids, etc. Following contact of the surface with the oxidizing solution, excess is removed as described above, e.g., in the presence of a gas that limits the evaporation of the solvent of the oxidizing agent as described

For example, in an exemplary oxidation protocol, a volatile solution of tetrahydrofuran (THF) and pyridine and water may be used to carry the oxidizing agent which may be iodine as described herein, e.g., 70% THF, 20% pyridine, 10% water and 0.2% iodine. The solution may be introduced into a flow cell in any suitable manner, e.g., introduced into a vertical flow cell from the bottom up and may be drained from the bottom as well. However, while the solution is draining and before a washing solution is introduced, evaporation of the solvent may occur resulting in localized and non-uniform over-oxidation. Embodiments include introducing a gas into the flow cell at least during the time the solution is draining, where the gas limits the evaporation of the THF solvent. The gas may be any suitable gas such as a gas that is inert to the oxidizing agent—in this case iodine, e.g., the gas may be nitrogen. The gas may include a second component that is the THF solvent mixture (may include all the components of the oxidizing liquid except the iodine), i.e., may include a vapor of the solvent. The gas may include a sufficient amount of the solvent such that the solvent is in equilibrium between the gas and any oxidizing liquid present on the surface of the substrate. In certain embodiments, the gas is saturated with the solvent as described above.

Optional Capping

In addition, unreacted hydroxyl groups may be (though not necessarily) capped, e.g., using any convenient capping agent, as is known in the art. This optional capping is accomplished by contacting the surface with an capping solution, as described above, which solution includes a suitable capping agent, such as a solution of acetic anhydride, pyridine or 2,6-lutidine (2,6-dimethylpyridine), and THF; a solution of 1-methyl-imidazole in THF; etc. Following contact of the surface with the capping solution, excess capping solution is removed as described above, e.g., in the presence of a gas that limits the evaporation of the solvent of the capping agent as described above.

For example, in an exemplary capping protocol, a solution of (THF) may be used to carry the capping agent which may be pyridine as described herein. The solution may be introduced into a flow cell in any suitable manner, e.g., introduced into a vertical flow cell from the bottom up and may be drained from the bottom as well. However, while the solution is draining and before a washing solution is introduced, evaporation of the solvent (e.g., THF) may occur resulting in localized and non-uniform excessive capping. Embodiments include introducing a gas into the flow cell at least during the time the solution is draining, where the gas limits the evaporation of the THF solvent. The gas may be any suitable gas such as a gas that is inert to the capping agent—in this case pyridine, e.g., the gas may be nitrogen. The gas may include a second component that is the THF solvent (may include all the components of the capping liquid except the pyridine), i.e., may include a vapor of the solvent. The gas may include a sufficient amount of the solvent such that the solvent is in equilibrium between the gas and any capping liquid present on the surface of the substrate. In certain embodiments, the gas is saturated with the solvent as described above.

Deblocking

The next substep in the subject methods is the deblocking step, where acid labile protecting groups present at the 5′ ends of the growing nucleic acid molecules on the substrate are removed to provide free 5′ OH moieties, e.g., for attachment of subsequent monomers, etc. In this deblocking step (which may also be referred to as a deprotecting step as results in removal of the protecting blocking groups), the entire substrate surface is contacted with a deblocking or deprotecting agent, typically in a flow cell, as described above. The substrate surface is incubated for a sufficient period of time under appropriate conditions for all available protecting groups to be cleaved from the nucleotides that they are protecting. Following contact of the surface with the deblocking solution, excess deblocking solution is removed as described above, e.g., in the presence of a gas that limits the evaporation of the solvent of the deblocking agent as described above.

In certain exemplary embodiments, the deblocking solution includes an acid present in an organic solvent, e.g., one that has a low vapor pressure. Organic solvents that may be used include, but are not limited to, toluene, xylene (o, m, p), ethylbenzene, perfluoro-n-heptane, perfluoro decalin, chlorobenzene, 1,2 dichloroethane, 1,1,2 trichloroethane, 1,1,2,2 tetrachloroethane, pentachloroethane, and the like; where in certain embodiments, the organic solvent that is employed is toluene. In yet other embodiments, the employed solvent is a methylbenenze solvent that has a higher moleculare weight that toluene, e.g., dimethylbenzene (xylene), trimethylbenzene, etc. The acid deblocking agent employed in the deblocking solution may vary, where representative acids include, but are not limited to: acetic acids, e.g., acetic acid, mono acetic acid, dichloroacetic acid, trichloroacetic acid, monofluoroacetic acid, difluoroacetic acid, trifluoroacetic acid, and the like. The amount of acid in the solution is sufficient to remove blocking groups, and may range from between about 0.1 and about 20%, e.g., from between about 1 and about 3%, as is known in the art.

Contact of the substrate surface with a deblocking solution results in removal of the protecting groups from the blocked substrate bound residues. As such, this step results in the deprotection of the nucleotide residues on the substrate surface. Following deprotection, the deblocking solution is removed from the surface of the substrate.

Removal of the deblocking agent according to the subject methods results in a substrate surface in which the nucleotide residues are deprotected. In others words, removal of the deblocking agent results in the production of an array of nucleotide residues stably associated with the substrate surface, where the nucleotide residues on the array surface have 5′-OH groups available for reaction with an activated nucleotide in subsequent cycles.

For example, in an exemplary deblocking protocol, a volatile solvent may be used to carry the deblocking agent, which deblocking agent may be for example a species of chloroacetic acid such as dichloroacetic acid or any agent described herein. For example, a deblocking liquid that includes a deblocking reagent such as dichloroacetic acid for example, in toluene for example may be flowed across a substrate surface positioned in a flow cell, where the substrate surface may display a first chemical moiety precursor. The deblocking solution may be introduced into a flow cell in any suitable manner, e.g., introduced into a vertical flow cell from the bottom up and may be drained from the bottom as well. However, while the solution is draining and before a washing solution is introduced, evaporation of the solvent may occur resulting in localized and non-uniform deblocking. Embodiments include introducing a gas into the flow cell at least during the time the deblocking solution is draining, where the gas limits the evaporation of the deblocking solvent, in this embodiments toluene solvent. The gas may be any suitable gas such as a gas that is inert to the deblocking agent—in this case dichloroacetic acid, e.g., the gas may be nitrogen. The gas may include a second component that is the toluene solvent (may include all the components of the deblocking liquid except the dichloroacetic acid), i.e., may include a vapor of the solvent. The gas may include a sufficient amount of the solvent such that the solvent is in equilibrium between the gas and any deblocking liquid present on the surface of the substrate. In certain embodiments, the gas is saturated with the solvent as described above.

A feature of certain embodiments the subject methods is that depurination reactions resulting from the increase in effective acid deblocking agent during evaporation of the solvent from the surface do not occur to any significant extent. Specifically, by limiting solvent evaporation during deblocking in a manner according to the present invention, depurination is reduced. A feature of the arrays produced according to these embodiments where depurination is reduced, if not eliminated, is that each probe location of the arrays is highly uniform in terms of probe composition, since substantially no depurination side reactions occur during the array processing, if any. As such, the proportion of full-length sequence within each feature is higher as compared to arrays produced using analogous protocols but not according to the present invention, as described herein (e.g., at least about 5-fold higher, often at least about 10-fold higher, such as at least about 25-, 50- or 75-fold higher), and the length distribution within each feature is less skewed towards shorter sequences. As a result, background noise and non-selective signal are reduced in the hybridization signal.

Washing

In exemplary embodiments, the surface of the substrate may be washed between one or more of the above described capping, oxidation and deblocking steps. Any convenient wash fluid may be employed in these one or more wash steps. In certain embodiments, the wash fluid may be a low viscosity fluid. In these embodiments, the viscosity of the wash fluid typically does not exceed about 1.2, and in certain embodiments does not exceed about 0.6, such as about 0.4 cP (as measured at 25° C.). In certain embodiments, the wash fluid is an organic solvent or an ionic liquid. In certain embodiments, solvents of from 1 to about 6, more usually from 1 to about 4, carbon atoms, including alcohols such as methanol, ethanol, propanol, etc., ethers such as tetrahydrofuran, ethyl ether, propyl ether, etc., acetonitrile, dimethylformamide, dimethylsulfoxide, and the like, may be employed. Specific organic solvents of interest include, but are not limited to: acetonitrile, acetone, methanol, ethanol and the like. Embodiments include flowing a wash fluid over a surface of a substrate in a flow cell, wherein the wash liquid is flowed across the surface at least partially in the presence of a gas that limits evaporation of the solvent of the wash fluid.

The amount of the reagents employed in each of the above steps in the method of the present invention is dependent on the nature of the reagents, solubility of the reagents, reactivity of the reagents, availability of the reagents, purity of the reagents, and so forth. Such amounts should be readily apparent to those skilled in the art in view of the disclosure herein. In one aspect, stoichiometric amounts are employed; however, in other aspects excess of one reagent over the other may be used where circumstances dictate. Typically, the amounts of the reagents are those necessary to achieve the overall synthesis of the chemical compound, which may be, e.g., a nucleic acid as described above, in accordance with the present invention.

After a reagent is introduced into a flow cell, the reagent is held in contact with the array substrate for a time and under conditions sufficient for the particular step to be completed. The time periods and conditions are dependent on the nature of the reagent and the nature of the particular step of the procedure. For example, the time periods and conditions may be different for a washing procedure rather than an oxidizing reaction or a deblocking reaction. In general, the time periods and conditions for the procedures conducted in the flow cells are well-known in the art and will not be repeated here.

Following the 5′-generation step, summarized above, the remaining fluid, e.g., wash fluid, may be removed from the surface, e.g., by draining, and the surface dried, e.g., in the presence of an evaporation-limiting gas.

The above steps of: (a) monomer attachment; and (b) functional group regeneration, e.g., 5′OH hydroxyl regeneration, are repeated a number of times with additional monomers, e.g., nucleotides until each of the desired polymers, e.g., nucleic acids on the substrate surface are produced. By choosing which sites are contacted with which activated nucleotides, e.g. A, G, C & T, an array having polymers of desired sequence and spatial location is readily achieved.

As such, the above cycles of monomer attachment and functional (e.g., hydroxyl) moiety regeneration result in the production of an array of desired polymers, e.g., nucleic acids. The resultant arrays can be employed in a variety of different applications, as described in greater detail below.

The subject invention has been described above in terms of fabrication of nucleic acids arrays. While the above description has been provided in terms of nucleic acid array production protocols for ease and clarity of description, the scope of the invention is not so limited, but instead extends to the fabrication of any type of array structure, particularly biopolymeric array structure, including, but not limited to polypeptide arrays, in addition to the above described nucleic acid arrays.

Array Manufacturing Apparatuses

Also provided are chemical array manufacturing apparatuses. Embodiments include apparatuses that include a chemical moiety precursor deposition element configured to deposit a chemical moiety precursor onto a surface of a substrate, a reaction chamber such as a flow cell for flowing a liquid across a surface of the substrate that includes a functional group generation agent in a solvent, and a reservoir in fluid communication with the reaction chamber, where the reservoir includes a gas that limits evaporation of the solvent of the functional group generation liquid.

In certain embodiments, an apparatus may include one or more stations such as a fluid deposition station, e.g., a fluid deposition station that may be separate from a reaction chamber such as a flow cell, and which may include a reservoir filled with blocked monomer, e.g, blocked nucleoside. In some aspects, a fluid dispensing station may be employed which may be in fluid communication with the substrate surface so that there is some connection through which fluid may flow between a dispensing station and a substrate surface. A substrate may be transported to and from various stations using, for example, a robotic arm or the like. In certain embodiments, the fluid deposition station may include a pulse jet deposition device.

In certain embodiments, an apparatus may include at least two stations such that synthesis may be carried out at least at two stations, a first station for monomer attachment, e.g., that includes a chemical moiety precursor deposition element, and a second station such as a flow cell station in which functional group generation protocols may be performed, where in certain embodiments a station for functional group generation may include one or more flow cells. When moving the substrate between stations such as between a station that includes a chemical moiety precursor deposition element and flow cell stations, the substrate may be transported by a transfer element such as a robotic arm, and so forth. In one embodiment, a transfer robot is mounted on a platform of an apparatus used in the synthesis. The transfer robot may include a base, an arm that is movably mounted on the base, and a grasping element adapted to grasp the substrate during transport that is attached to the arm. The element for grasping the substrate may be, for example, movable finger-like projections, and the like. In one aspect, in use, the robotic arm is activated so that the substrate is grasped by the grasping element. The arm of the robot is moved so that the substrate is delivered to the flow cell.

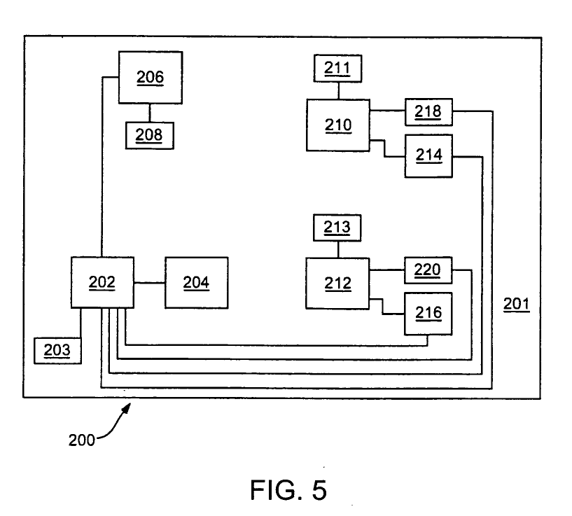

One embodiment of an apparatus that includes one or more flow cell assemblies in accordance with the present invention is depicted in FIG. 5 in schematic form. Apparatus 200 comprises platform 201 on which the components of the apparatus are mounted. Apparatus 200 includes main computer 202, with which various components of the apparatus are in communication. Video display 203 is in communication with computer 202. Apparatus 200 further includes print chamber 204, which is controlled by main computer 202. The nature of print chamber 204 depends on the nature of the printing technique employed to add monomers to a growing polymer chain. Such printing techniques include, by way of illustration and not limitation, pulse-jet deposition printing, and so forth.

Transfer robot 206 is also controlled by main computer 202 and includes a robot arm 208 that moves a substrate to be printed from print chamber 204 to either first flow cell assembly 210 or second flow cell assembly 212 (or to any other position such as to and/or from a printing chamber). Robot arm 208 may introduce a substrate into print chamber 204 horizontally or vertically for printing on a surface of the substrate and may introduce the substrate into a flow cell horizontally or vertically.

First flow cell assembly 210 is in communication with program logic controller 214 (which corresponds to a controller (not shown), which is controlled by main computer 202, and second flow cell 212 is in communication with program logic controller 216, which is also controlled by main computer 202. First flow cell 210 assembly is in communication with fluid dispensing station 211 and flow sensor and level indicator 218, which are controlled by main computer 202, and second flow cell assembly 212 is in communication with fluid dispensing station 213 and flow sensor and level indicator 220, which are also controlled by main computer 202.

One or both flow cells may also be in communication with a reservoir of gas (not shown), wherein the reservoir includes a gas that limits evaporation of the solvent of a liquid flowed in the flow cell, as described above.

The apparatus of the invention further includes appropriate electrical and mechanical architecture and electrical connections, wiring and devices such as timers, clocks, and so forth for operating the various elements of the apparatus. Such architecture is familiar to those skilled in the art and will not be discussed in more detail herein.

The methods in accordance with the present invention may be carried out under computer control, that is, with the aid of a computer. For example, an IBM® compatible personal computer (PC) may be utilized. The computer may be driven by software specific to the methods described herein. Computer hardware capable of assisting in the operation of the methods in accordance with the present invention involves in certain embodiments a system with at least the following specifications: Pentium® processor or better with a clock speed of at least 100 MHz, at least 32 megabytes of random access memory (RAM) and at least 80 megabytes of virtual memory, running under either the Windows 95 or Windows NT 4.0 operating system (or successor thereof). Software that may be used to carry out the methods may be, for example, Microsoft Excel or Microsoft Access, suitably extended via user-written functions and templates, and linked when necessary to stand-alone programs. Examples of software or computer programs used in assisting in conducting the present methods may be written, preferably, in Visual BASIC, FORTRAN and C++. It should be understood that the above computer information and the software used herein are by way of example and not limitation. The present methods may be adapted to other computers and software. Other languages that may be used include, for example, PASCAL, PERL or assembly language.

A computer program may be utilized to carry out the above method steps. The computer program provides for controlling the valves of the flow assemblies to introduce reagents into the flow cells, vent the flow cells, and so forth. The computer program may provide for controlling valves of a gas reservoir to introduce a suitable evaporation-limiting gas to a flow cell. The computer program further may provide for moving the substrate to and from a station for monomer addition at a predetermined point in the aforementioned method.

Another aspect of the present invention is a computer program product including a computer readable storage medium having a computer program stored thereon which, when loaded into a computer, performs the aforementioned method.

In exemplary embodiments, the methods are coded onto a computer-readable medium in the form of programming.

The data storage means may include any manufacture including a recording of the present information as described above, or a memory access means that can access such a manufacture.

In certain embodiments, a processor of the subject invention may be in operable linkage, i.e., part of or networked to, the aforementioned device, and capable of directing its activities.

A processor may be pre-programmed, e.g., provided to a user already programmed for performing certain functions, or may be programmed by a user, where a processor may be programmed, e.g., by a user, from a remote location meaning a location other than the location at which the processor and/or flow cell and/or substrate is present. For example, a remote location could be another location (e.g. office, lab, etc.) in the same city, another location in a different city, another location in a different state, another location in a different country, etc. A processor may be remotely programmed by “communicating” programming information to the processor, i.e., transmitting the data representing that information as electrical signals over a suitable communication channel (for example, a private or public network). “Forwarding” programming refers to any means of getting that programming from one location to the next, whether by physically transporting that programming or otherwise (where that is possible) and includes, physically transporting a medium carrying the programming or communicating the programming. Any convenient telecommunications means may be employed for transmitting the programming, e.g., facsimile, modem, Internet, LAN, WAN or other network means, etc.

Chemical Arrays

Also provided by the subject invention are arrays of nucleic acids produced according to the subject methods, as described above. The subject arrays include at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g., as a spot on the surface of the substrate. The number of distinct nucleic acid sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm2, where the density may be as high as 106 or higher, but will generally not exceed about 105 spots/cm2. In other embodiments, the polymeric sequences are not arranged in the form of distinct spots, but may be positioned on the surface such that there is substantially no space separating one polymer sequence/feature from another.

As indicated above, the arrays are arrays of nucleic acids, including oligonucleotides, polynucleotides, DNAs, RNAs, synthetic mimetics thereof, and the like.

Utility

Chemical arrays produced according to the subject methods find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992.

In certain embodiments, the subject methods include a step of transmitting data from at least one of the detecting and deriving steps, as described above, to a remote location.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. patent application Ser. No. 09/846,125 “Reading Multi-Featured Arrays” by Dorsel et al.; and Ser. No. 09/430,214 “Interrogating Multi-Featured Arrays” by Dorsel et al. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of contacting a surface of an object with an agent, said method comprising:
(a) positioning said object in a reaction chamber; and
(b) flowing a liquid comprising said agent in a solvent across said surface of said object in said reaction chamber, wherein said liquid is flowed across said surface at least partially in the presence of a gas that limits evaporation of said solvent;
thereby contacting said surface of said object with said agent.

2. The method according to claim 1, wherein said gas comprises at least two components, wherein a first component is a gas inert to said agent.

3. The method according to claim 2, wherein said gas comprises a second component that is said solvent.

4. The method according to claim 3, wherein said gas comprises a sufficient amount of said solvent such that said solvent is in equilibrium between said gas and any liquid present on said surface of said object.

5. The method according to claim 4, wherein said solvent is a volatile solvent.

6. The method according to claim 5, wherein said volatile solvent has a vapor pressure of at least about 0.3 mm.

7. The method according to claim 6, wherein said solvent is an organic solvent.

8. The method according to claim 3, wherein said gas is saturated with respect to said solvent.

9. The method according to claim 1, wherein said reaction chamber is a flow cell.

10. The method according to claim 9, wherein said flowing comprises introducing a volume of said liquid into and then removing said volume of liquid from said flow cell.

11. The method according to claim 10, wherein said gas is present at least during said removing portion of said flowing step.

12. A method of producing a chemical array, said method comprising:
(a) positioning a substrate having a surface displaying a first chemical moiety precursor in a reaction chamber; and
(b) flowing a liquid comprising a functional group generation reagent in a solvent across said surface in said reaction chamber to generate a functional group on said first chemical moiety, wherein said liquid is flowed across said surface at least partially in the presence of a gas that limits evaporation of said solvent;
to produce said chemical array.

13. The method according to claim 12, wherein said method further comprises reacting said first chemical moiety displaying a functional group produced by step (b) with a second chemical moiety precursor.

14. The method according to claim 13, wherein said reacting occurs by depositing said second chemical moiety precursor onto said surface.

15. The method according to claim 15, wherein said depositing occurs outside of said reaction chamber.

16. The method according to claim 12, wherein said chemical array is a polymeric array.

17. The method according to claim 16, wherein said polymeric array is a peptide array.

18. The method according to claim 16, wherein said polymeric array is nucleic acid array.

19. The method according to claim 12, wherein said first and second chemical moiety precursors are blocked monomers.

20. The method according to claim 19, wherein said functional group generation reagent is a deblocking agent.

21. The method according to claim 12, wherein said reaction chamber is a flow cell.

22. A method of producing a nucleic acid array, said method comprising:
(a) positioning a substrate in a flow cell, wherein said substrate comprises a surface displaying a covalently bound blocked nucleotide;
(b) flowing a liquid comprising a deblocking reagent in a solvent across said surface in said flow cell to deblock said bound nucleotide, wherein said liquid is flowed across said surface by introducing a volume of said liquid into said flow cell and then removing said volume of liquid from said flow cell, wherein at least said removing step occurs in the presence of a gas that limits evaporation of said solvent;
(c) contacting said bound deblocked nucleotide with a blocked nucleoside to covalently bond said second blocked nucleoside to said bound deblocked nucleotide; and
(d) reiterating steps (a) and (b) at least once to produce said nucleic acid array.

23. The method of claim 22, wherein said method comprises sequentially flowing a plurality of different liquids across said surface in said flow cell.

24. The method of claim 23, wherein said plurality of different fluids includes at least an oxidizing liquid and a deblocking liquid.

25. The method of claim 24, wherein said plurality of different fluids further includes a wash fluid.

26. The method of claim 22, wherein said blocked nucleoside is contacted with said surface by pulse-jet deposition.

27. An apparatus for producing a chemical array, said apparatus comprising:
(a) a chemical moiety precursor deposition element for depositing a chemical moiety precursor onto a surface of a substrate;
(b) a reaction chamber for flowing a liquid across a surface of a substrate in communication with a reservoir of said liquid, wherein said liquid comprises a functional group generation agent in a solvent; and
(c) a reservoir of a gas in fluid communication with said reaction chamber, wherein said reservoir comprises a gas that limits evaporation of said solvent.

28. The apparatus according to claim 27, wherein said gas comprises at least two components, wherein a first component is a gas inert to said agent.

29. The apparatus according to claim 28, wherein said gas comprises a second component that is said solvent.

30. The apparatus according to claim 27, wherein said solvent is a volatile solvent.

31. The apparatus according to claim 30, wherein said volatile solvent has a vapor pressure of at least about 0.3 mm.

32. The apparatus according to claim 31, wherein said solvent is an organic solvent.

33. The apparatus according to claim 29, wherein said gas is saturated with respect to said solvent.

34. The apparatus according to claim 27, wherein said reaction chamber is a flow cell.

35. The apparatus according to claim 27, wherein said apparatus further comprises a fluid deposition station.

36. The apparatus according to claim 36, wherein said fluid deposition station is separate from said reaction chamber.

37. The apparatus according to claim 36, wherein said fluid deposition station comprises a pulse-jet deposition device.

38. The apparatus according to claim 36, wherein said fluid deposition station comprises a reservoir filled with a blocked monomer.

39. The apparatus according to claim 38, wherein said blocked monomer is a blocked nucleoside.
